(19) United States
(12) Patent Application Publication
(10) Pub. No.: US 2022/0211297 A1
(43) Pub. Date: Jul. 7, 2022

(54) INFORMATION PROCESSING DEVICE, WALKING ENVIRONMENT DETERMINATION DEVICE, WALKING ENVIRONMENT DETERMINATION SYSTEM, INFORMATION PROCESSING METHOD, AND STORAGE MEDIUM

(71) Applicant: NEC Corporation, Minato-ku, Tokyo (JP)

(72) Inventors: Chenhui HUANG, Tokyo (JP); Kenichiro FUKUSHI, Tokyo (JP); Kazuki IHARA, Tokyo (JP); Noriyuki TONOUCHI, Tokyo (JP)

(73) Assignee: NEC Corporation, Minato-ku, Tokyo (JP)

(21) Appl. No.: 17/611,649

(22) PCT Filed: May 29, 2019
(86) PCT No.: PCT/JP2019/021420

§ 371 Date: Nov. 16, 2021

(51) Int. Cl. A61B 5/11; G01P 13/00; G01P 15/08; G01P 3/00; G01P 15/18; A61B 5/00

(52) U.S. Cl. CPC A61B 5/112; G01P 13/00; G01P 15/08; A61B 2562/0219; G01P 15/18; A61B 5/6807; G01P 3/00

(57) ABSTRACT

Provided is an information processing device including an acquisition unit configured to acquire motion information of a foot of a user measured by a motion measurement device provided on the foot and a feature amount extracting unit configured to extract a feature amount indicating a walking environment from an angle between a sole and a ground generated based on the motion information.

### TECHNICAL FIELD

The present invention relates to an information processing device, a walking environment determination device, a walking environment determination system, an information processing method, and a storage medium.

### BACKGROUND ART

Patent Literature 1 discloses a system for measuring a walking state using motion data and floor reaction force data. Patent Literature 2 discloses a system for measuring a walking state using three-dimensional angle information of a knee or the like.

### CITATION LIST

### Patent Literature

PTL 1: International Publication No. WO2018/101071
PTL 2: Japanese Patent Application Laid-open No. 2017-144237

### Non Patent Literature

NPL 1: Sebastian O. H. Madgwick, Andrew J. L. Harrison, and Ravi Vaidyanathan, “Estimation of IMU and MARG orientation using a gradient descent algorithm,” 2011 IEEE International Conference on Rehabilitation Robotics, pp. 179-185, 2011.

### SUMMARY OF INVENTION

### Technical Problem

In connection with the measurement of the walking state, a determination of a walking environment such as whether or not a location where the user is walking is level ground may be further required. In the method for measuring a walking state as disclosed in Patent Literature 1 or Patent Literature 2, depending on elements used for determining a walking state, a walking environment may not be accurately determined.

The present invention intends to provide an information processing device, a walking environment determination device, a walking environment determination system, an information processing method, and a storage medium which can suitably extract a feature amount used for determining a walking environment.

### Solution to Problem

According to one example aspect of the invention, provided is an information processing device including an acquisition unit configured to acquire motion information of a foot of a user measured by a motion measurement device provided on the foot and a feature amount extracting unit configured to extract a feature amount indicating a walking environment from an angle between a sole and a ground generated based on the motion information.

According to another example aspect of the invention, provided is an information processing method including acquiring motion information of a foot of a user measured by a motion measurement device provided on the foot and extracting a feature amount indicating a walking environment from an angle between a sole and a ground generated based on the motion information.

According to another example aspect of the invention, provided is a storage medium storing a program that causes a computer to perform acquiring motion information of a foot of a user measured by a motion measurement device provided on the foot and extracting a feature amount indicating a walking environment from an angle between a sole and a ground generated based on the motion information.

### Advantageous Effects of Invention

According to the present invention, an information processing device, a walking environment determination device, a walking environment determination system, an information processing method, and a storage medium which can suitably extract a feature amount used for determining a walking environment can be provided.

### BRIEF DESCRIPTION OF DRAWINGS

FIG. 1 is a schematic diagram illustrating a general configuration of a walking environment determination system according to a first example embodiment.

FIG. 2 is a block diagram illustrating a hardware configuration of a walking environment determination device according to the first example embodiment.

FIG. 3 is a block diagram illustrating a hardware configuration of an information communication terminal according to the first example embodiment.

FIG. 4 is a functional block diagram of an information processing device according to the first example embodiment.

FIG. 5 is a flowchart illustrating an example of walking environment determination processing performed by the walking environment determination device according to the first example embodiment.

FIG. 6 is a conceptual diagram illustrating a walking cycle.

FIG. 7 is a graph illustrating an example of time series data of the angle between the sole and the ground in one walking cycle.

FIG. 8 is a graph illustrating an example of the angle between the sole and the ground at the time of landing.

FIG. 9 is a graph illustrating an example of time series data of acceleration in a vertical direction in one walking cycle.

FIG. 10 is a flowchart illustrating an example of training process performed by the server according to the first example embodiment.

FIG. 11 is a table schematically illustrating the correspondence relation between a feature amount vector acquired and a walking state label by a training process.

FIG. 12 is a table illustrating a result of cross-validation.

FIG. 13 is a functional block diagram of an information processing device according to a second example embodiment.

FIG. 14 is a flowchart illustrating an example of a mode switching process performed by the walking environment determination device according to the second example embodiment.

FIG. 15 is a functional block diagram of an information processing device according to a third example embodiment.

### DESCRIPTION OF EMBODIMENTS

Exemplary embodiments of the present invention are described below with reference to the drawings. Throughout the drawings, the same components or corresponding components are labeled with same references, and the description thereof may be omitted or simplified.

### First Example Embodiment

A walking environment determination system according to the present example embodiment is described. The walking environment determination system of the present example embodiment is a system for measuring and analyzing a walking state including determination of a walking environment of a user. In this specification, the “walking environment” means a state of the ground where the user is walking. More specifically, the “walking environment” refers to, for example, a location where the user is walking is level ground or a location other than level ground such as stairs or a slope. The “walking state” includes, in addition to the walking environment, a feature included in the walking pattern of the user (gait).

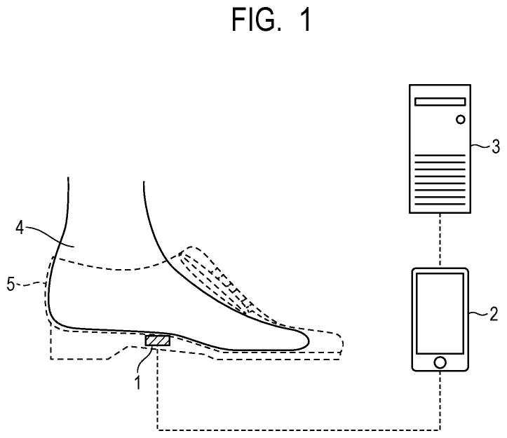

FIG. 1 is a schematic diagram illustrating a general configuration of a walking environment determination system according to the present example embodiment. The walking environment determination system includes a walking environment determination device 1, an information communication terminal 2, and a server 3 which can be connected to each other by wireless communication.

The walking environment determination device 1 is provided to be close to the sole of a shoe 5 worn by a user 4, for example. The walking environment determination device 1 is an electronic apparatus having a sensing function for measuring a motion of the foot of the user 4, an information processing function for analyzing the measured motion information, a communication function with the information communication terminal 2, and the like. It is desirable that the walking environment determination device 1 be provided at a position corresponding to the arch of the foot such as just below the arch of the foot. In this case, the walking environment determination device 1 can measure acceleration and angular velocity of the center of the foot of the user 4. Since the center of the foot is a position showing the feature of the motion of the foot well, it is suitable for feature amount extraction.

Note that, the walking environment determination device 1 may be provided in the insole of the shoe 5, may be provided in the outsole of the shoe 5, or may be embedded in the shoe 5. The walking environment determination device 1 may be detachably attached to the shoe 5 or may be non-detachably fixed to the shoe 5. The walking environment determination device 1 may be provided at a portion other than the shoe 5 as long as the walking environment determination device 1 can measure the motion of the foot. For example, the walking environment determination device 1 may be provided in a sock which the user 4 is wearing, provided in a decoration, directly attached to the foot of the user 4, or embedded in the foot of the user 4. Although FIG. 1 illustrates an example in which one walking environment determination device 1 is provided on one foot of the user 4, one walking environment determination device 1 may be provided on each of both feet of the user 4. In this case, the motion information of both feet can be acquired in parallel, and more information can be acquired.

In this specification, the “foot” means a body part below an ankle of the user 4. In addition, in this specification, the “user” means a person who is an object of a determination of the walking environment using the walking environment determination device 1. Whether or not the user corresponds to the “user” is unrelated to whether or not the user is a user of a device other than the walking environment determination device 1 constituting the walking environment determination system, whether or not the user receives a service provided by the walking environment determination system, or the like.

The information communication terminal 2 is a terminal device carried by the user 4, such as a cellular phone, a smartphone, or a smart watch. Application software for analyzing a walking state is installed in advance in the information communication terminal 2, and processing based on the application software is performed. The information communication terminal 2 acquires data such as the determination result of the walking environment and the walking state acquired by the walking environment determination device 1 from the walking environment determination device 1 and performs information processing using the data. The result of the information processing may be notified to the user 4 or may be transmitted to the server 3. The information communication terminal 2 may have a function of providing software such as a control program of the walking environment determination device 1 or a data analysis program to the walking environment determination device 1.

The server 3 provides application software for analyzing walking states to the information communication terminal 2 and updates the application software. The server 3 may store data acquired from the information communication terminal 2 and perform information processing using the data.

Note that, the general configuration is an example, and for example, the walking environment determination device 1 may be directly connected to the server 3. Further, the walking environment determination device 1 and the information communication terminal 2 may be configured as an integrated device, and another device such as an edge server or a relay device may be further included in the walking environment determination system.

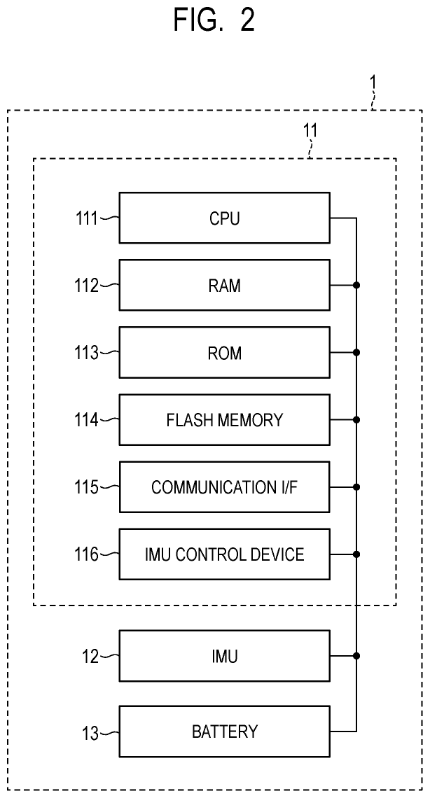

FIG. 2 is a block diagram illustrating a hardware configuration example of the walking environment determination device 1. The walking environment determination device 1 includes an information processing device 11, an inertial measurement unit (IMU) 12, and a battery 13.

The information processing device 11 is, for example, a microcomputer or a microcontroller that performs a control and data processing of the entire walking environment determination device 1. The information processing device 11 includes a central processing unit (CPU) 111, a random access memory (RAM) 112, a read only memory (ROM) 113, a flash memory 114, a communication interface (I/F) 115, and an IMU control device 116. Each unit in the information processing device 11, the IMU 12, and the battery 13 is connected each other via a bus, wiring, a driving device, or the like.

The CPU 111 is a processor that performs predetermined calculation in accordance with a program stored in the ROM 113, the flash memory 114, or the like, and also has a function of controlling each unit of the information processing device 11. The RAM 112 is composed of a volatile storage medium and provides a temporary memory area required for the operation of the CPU 111. The ROM 113 is composed of a non-volatile storage medium and stores necessary information such as a program used for the operation of the information processing device 11. The flash memory 114 is a storage device composed of a non-volatile storage medium and temporarily storing data, storing an operation program of the information processing device 11, or the like.

The communication I/F 115 is a communication interface based on standards such as Bluetooth (registered trademark) or Wi-Fi (registered trademark), and is a module for performing communication with the information communication terminal 2.

The IMU 12 is a motion measurement device including an angular velocity sensor that measures angular velocity in three axial directions and an acceleration sensor that measures acceleration in three directions. The angular velocity sensor may be any sensor as long as it can acquire the angular velocity as time series data, and any type of sensor such as a vibration type sensor or a capacitance type sensor may be used. The acceleration sensor may be any type of sensor as long as it can acquire acceleration as time series data, and any type of sensor such as a piezoelectric type sensor, a piezoresistance type sensor, or a capacitance type sensor may be used. In the present example embodiment, the interval between the data points of the acquired time series data may be constant or may not be constant.

The IMU control device 116 is a control device that controls the IMU 12 to measure angular velocity and acceleration and acquires angular velocity and acceleration acquired by the IMU 12. The acquired angular velocity and acceleration are stored in the flash memory 114 as digital data. Note that analog-to-digital (AD) conversion for converting an analog signal measured by the IMU 12 into digital data may be performed in the IMU 12 or may be performed by the IMU control device 116.

The battery 13 is, for example, a secondary battery, and supplies power necessary for the operations of the information processing device 11 and the IMU 12. Since the battery 13 is built in the walking environment determination device 1, the walking environment determination device 1 can operate wirelessly without connecting to an external power source by wire.

Note that the hardware configuration illustrated in FIG. 2 is an example, and other devices may be added or some devices may not be provided. Further, some devices may be replaced by other devices having similar functions. For example, the information processing device 11 may further include an input device such as a button so that an operation by the user 4 can be accepted, and may further include an output device such as a display, a display lamp, and a speaker for providing information to the user 4. Thus, the hardware configuration illustrated in FIG. 2 can be changed appropriately.

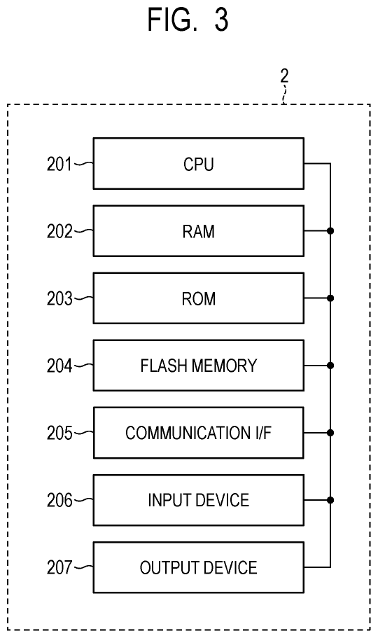

FIG. 3 is a block diagram illustrating a hardware configuration example of the information communication terminal 2. The information communication terminal 2 includes a CPU 201, a RAM 202, a ROM 203, and a flash memory 204. The information communication terminal 2 also includes a communication I/F 205, an input device 206, and an output device 207. Each unit of the information communication terminal 2 is connected to each other via a bus, wiring, a driving device, or the like.

In FIG. 3, each unit constituting the information communication terminal 2 is illustrated as an integrated device, but a part of these functions may be provided by an external device. For example, the input device 206 and the output device 207 may be external devices different from those constituting the functions of the computer including the CPU 201 or the like.

The CPU 201 is a processor that performs predetermined calculation in accordance with a program stored in the ROM 203, the flash memory 204, or the like, and also has a function of controlling each unit of the information communication terminal 2. The RAM 202 is composed of a volatile storage medium and provides a temporary memory area required for the operation of the CPU 201. The ROM 203 is composed of a non-volatile storage medium and stores necessary information such as a program used for the operation of the information communication terminal 2. The flash memory 204 is a storage device composed of a non-volatile storage medium for storing data transmitted and received to and from the walking environment determination device 1 and for storing a program for operating the information communication terminal 2.

The communication I/F 205 is a communication interface based on standards such as Bluetooth (registered trademark), Wi-Fi (registered trademark), or 4G and is a module for performing communication with other devices.

The input device 206 is a user interface used by the user 4 to operate the information communication terminal 2. Examples of the input device 206 include a mouse, a trackball, a touch panel, a pen tablet, a button, or the like.

The output device 207 is, for example, a display device. The display device is a liquid crystal display, an organic light emitting diode (OLED) display, or the like, and is used for displaying information, displaying a graphical user interface (GUI) for operation input, or the like. The input device 206 and the output device 207 may be integrally formed as a touch panel.

Note that the hardware configuration illustrated in FIG. 3 is an example, and other devices may be added or some devices may not be provided. Further, some devices may be replaced by other devices having similar functions. Further, some functions of the present example embodiment may be provided by other devices via a network, or some functions of the present example embodiment may be realized by being distributed among a plurality of devices. For example, the flash memory 204 may be replaced by a hard disk drive (HDD) or a cloud storage. Thus, the hardware configuration illustrated in FIG. 3 can be changed appropriately.

The server 3 is a computer having substantially the same hardware configuration as that illustrated in FIG. 3. Since the hardware configuration of the server 3 is substantially the same as that of the information communication terminal 2 except that the server 3 may not be portable, a detailed description thereof is omitted.

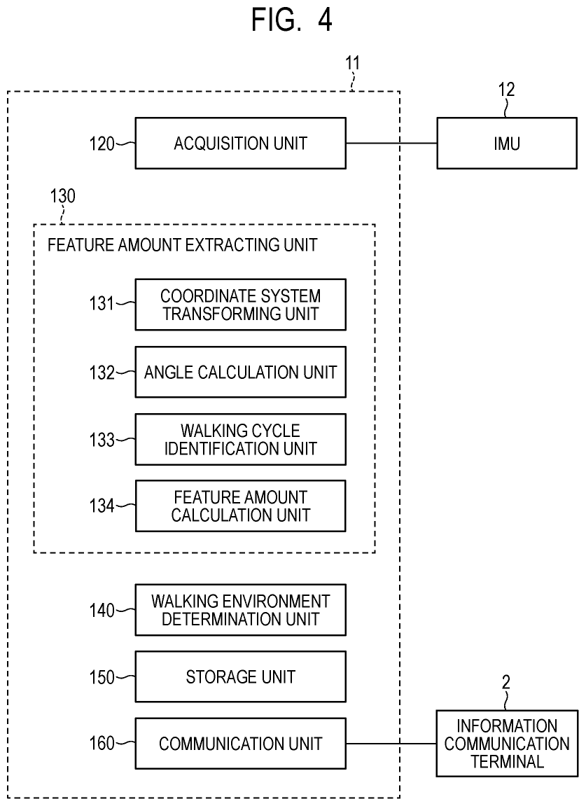

FIG. 4 is a functional block diagram of the information processing device 11 according to the present example embodiment. The information processing device 11 includes an acquisition unit 120, a feature amount extracting unit 130, a walking environment determination unit 140, a storage unit 150, and a communication unit 160. The feature amount extracting unit 130 includes a coordinate system transforming unit 131, an angle calculation unit 132, a walking cycle identification unit 133, and a feature amount calculation unit 134.

The CPU 111 loads a program stored in the ROM 113, the flash memory 114, or the like into the RAM 112 and executes the program. Thus, the CPU 111 realizes the functions of the feature amount extracting unit 130 and the walking environment determination unit 140. Further, the CPU 111 realizes the function of the acquisition unit 120 by controlling the IMU control device 116 based on the program. The CPU 111 realizes the function of the storage unit 150 by controlling the flash memory 114 based on the program. Further, the CPU 111 realizes the function of the communication unit 160 by controlling the communication I/F 115 based on the program. Specific processing performed by these units is described later.

In the present example embodiment, each function of the functional blocks illustrated in FIG. 4 is provided in the walking environment determination device 1, but some functions of the functional blocks illustrated in FIG. 4 may be provided in the information communication terminal 2 or the server 3. That is, the above-described functions may be realized by any of the walking environment determination device 1, the information communication terminal 2, and the server 3, or may be realized by cooperation of the walking environment determination device 1, the information communication terminal 2, and the server 3.

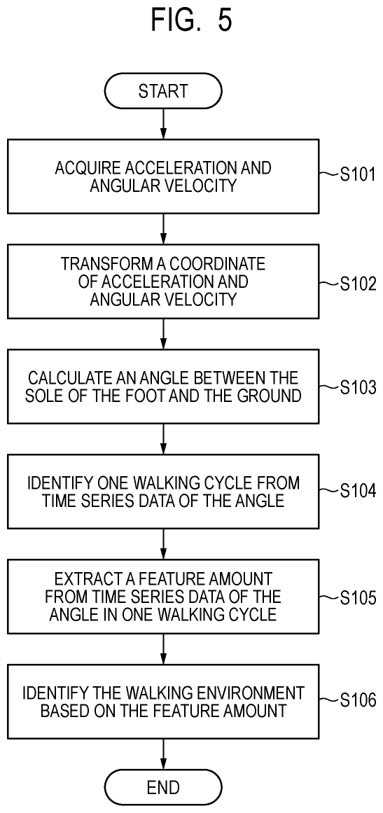

FIG. 5 is a flowchart illustrating an example of walking environment determination processing performed by the walking environment determination device 1 according to the present example embodiment. The process of FIG. 5 is performed when the walking environment determination device 1 detects walking, for example, when the user 4 is walking. Alternatively, the process of FIG. 5 may be always performed unrelated to whether or not the user 4 is walking, or may be performed at predetermined time intervals.

In step S101, the acquisition unit 120 controls the angular velocity sensor and the acceleration sensor of the IMU 12 to acquire time series data of angular velocity in three axial direction and acceleration in three directions. Thus, the acquisition unit 120 can acquire time changes in angular velocity and acceleration caused by walking of the user 4. The acquired time series data of angular velocity and acceleration is converted into digital data and then stored in the storage unit 150. These angular velocity and acceleration are referred to more generally as motion information. Among the motion information, time series data of angular velocity and acceleration during walking is particularly referred to as walking data. The walking data can be used not only for the determination of the walking environment of the present example embodiment but also for the gait analysis of the user 4.

The three directions of acceleration acquired by the acquisition unit 120 may be, for example, the width direction (left/right direction), the longitudinal direction (front/back direction), and the vertical direction of the foot of the user 4 provided with the IMU 12. These directions are referred to as x-axis, y-axis, and z-axis, respectively. The three axial directions of the angular velocity acquired by the acquisition unit 120 may be, for example, an adduction and an abduction of the foot about the z-axis (yaw), a pronation and a supination of the foot about the y-axis (pitch), and a bending and a stretching of the foot about x-axis (roll).

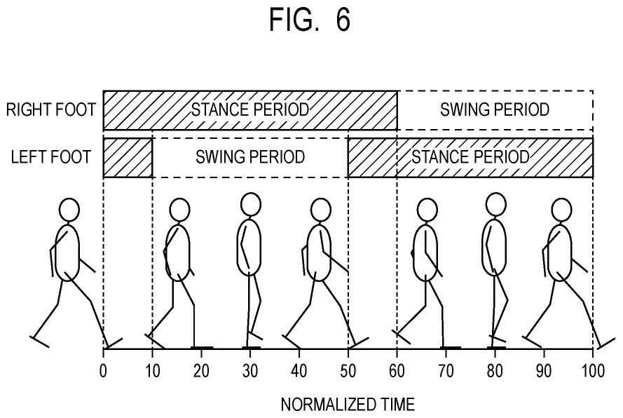

Here, in order to sufficiently acquire features included in walking, it is desirable that time series data of angular velocity in three axial directions and acceleration in three directions include data of a period corresponding to at least one walking cycle. One walking cycle is described with reference to FIG. 6. FIG. 6 is a conceptual diagram illustrating a walking cycle. FIG. 6 schematically illustrates motion of the right foot and the left foot of the user 4 for one walking cycle. The normalized time in the figure indicates the time normalized so that the length of one walking cycle is 100. That is, the normalized time 0 in the figure is the moment at which the right foot lands, the normalized time 50 in the figure is the moment at which the left foot lands, and the normalized time 100 in the figure is the moment at which the right foot lands again. A period from the normalized time 0 to 100 is one walking cycle.

Further, a period in which the foot lands is referred to as a stance period, and a period in which the foot leaves the ground is referred to as a swing period. More specifically, for example, the stance period of the right foot is a period from the moment at which the heel of the right foot lands (at the time of landing) to the moment at which the toe of the right foot leaves the ground (at the time of leaving), and generally occupies a period of about 60% of one walking cycle. The swing period of the right foot is a period from the moment when the toe of the right foot leaves the ground to the moment when the heel of the right foot lands, and generally occupies a period of about 40% of one walking cycle. As illustrated in FIG. 6, during walking, the stance period and the swing period are alternately repeated. Further, the phase of the stance period and the phase of the swing period are opposite between the right foot and the left foot.

In step S102, the coordinate system transforming unit 131 performs coordinate system transformation of angular velocity in three axial directions and acceleration in three directions. A coordinate system with respect to angular velocity and acceleration output by the IMU 12 is an inertial coordinate system. The coordinate system transforming unit 131 transforms the angular velocity and acceleration coordinate system into a coordinate system with respect to the foot of the user 4. Thus, the coordinate system of the angular velocity and the acceleration can be made suitable for calculating the angle between the sole and the ground. The transformation of the coordinate system is realized, for example, by multiplying the base vector of the inertial coordinate system by the direction cosine matrix E using the Euler angle and rotating the base vector.

An example of transformation of the coordinate system by the direction cosine matrix E is described more specifically. In a case where the base vector of the inertial coordinate system is [xᵢ, yᵢ, zᵢ], and the base vector of the coordinate system with respect to the foot is [x_{b}, y_{b}, z_{b}], a conversion formula between them is expressed by the following equation (1).

$$\begin{matrix}[ Math . 1 ] & \\ [ \begin{matrix}{x}_{b} \\ {y}_{b} \\ {z}_{b}\end{matrix} ] = [ \begin{matrix}{E}_{11} & {E}_{12} & {E}_{13} \\ {E}_{21} & {E}_{22} & {E}_{23} \\ {E}_{31} & {E}_{32} & {E}_{33}\end{matrix} ] [ \begin{matrix}{x}_{i} \\ {y}_{i} \\ {z}_{i}\end{matrix} ] = E [ \begin{matrix}{x}_{i} \\ {y}_{i} \\ {z}_{i}\end{matrix} ] & ( 1 )\end{matrix}$$

In a case where angle acquired by rotating the base vector of the inertial coordinate system by angles of ψ (psi), θ (theta), and φ (phi) in the order of z, y, and x is an Euler angle of the coordinate system transformation, the direction cosine matrix E is expressed by the following equation (2).

$$\begin{matrix}[ Math . 2 ] & \\ E = [ \begin{matrix}1 & 0 & 0 \\ 0 & cos ϕ & sin ϕ \\ 0 & - sin ϕ & cos ϕ\end{matrix} ] [ \begin{matrix}cos θ & 0 & - s in θ \\ 0 & 1 & 0 \\ sin θ & 0 & cos θ\end{matrix} ] [ \begin{matrix}cos ψ & sin ψ & 0 \\ - s in ψ & cos ψ & 0 \\ 0 & 0 & 1\end{matrix} ] = [ \begin{matrix}cos θ cos ψ & cos θ sin ψ & - s in θ \\ sin ϕ sin θ cos ψ - cos ϕ sin ψ & sin ϕ sin θ sin ψ + cos ϕ cos ψ & sin ϕ cos θ \\ cos ϕ sin θ cos ψ + sin ϕ sin ψ & cos ϕ sin θ sin ψ - sin ϕ cos ψ & cos ϕ cos θ\end{matrix} ] & ( 2 )\end{matrix}$$

Note that the calculation method used for conversion of the coordinate system is merely an example, and other calculation methods may be used. For example, a calculation method using a quaternion may be applied.

In step S103, the angle calculation unit 132 calculates the angle between the sole of the user 4 and the ground from the angular velocity in the three axial directions and the acceleration in the three directions after being transformed into the coordinate system with respect to the foot of the user 4. As a specific example of this process, there is a method in which angular velocity in three axial directions and acceleration in three directions are input to a Madgwick filter (Non Patent Literature 1), and a rotation angle in three axial directions of the foot is output as an output. The rotation angles in the three axial directions acquired by the Madgwick filter are the angles of adduction or abduction of the foot, the angle of pronation or supination of the foot, and the angle of bending or stretching of the foot. Out of these three angles, the angle of bending or stretching of the foot corresponds to the angle between the sole of the user 4 and the ground.

In step S104, the walking cycle identification unit 133 identifies one walking cycle from time series data including at least the angle between the sole of the user 4 and the ground. Since substantially the same motion is repeated for each step during walking, one walking cycle can be identified by detecting periodicity of time series data. For example, one walking cycle can be identified based on the appearance time of the peak or dip of the time series data, the frequency of the peak included in the frequency spectrum acquired by Fourier-transforming the time series data, or the like.

In step S105, the feature amount calculation unit 134 extracts a feature amount indicating the walking environment from the time series data of at least one walking cycle. The extracted feature amount is stored in the storage unit 150. The extraction of the feature amount is described with a specific example.

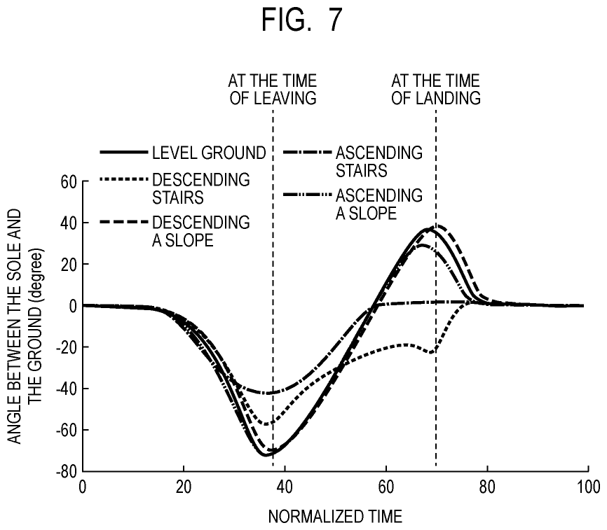

FIG. 7 is a graph illustrating an example of time series data of the angle between the sole and the ground in one walking cycle. The horizontal axis of FIG. 7 represents the normalized time in one walking cycle, and the vertical axis of FIG. 7 represents the angle between the sole and the ground. In FIG. 7, since the start point and the end point of one walking cycle are different from those in FIG. 6, the value of the normalized time does not coincide with that in FIG. 6. The sign of the angle is defined as positive when the toe of the foot faces upward.

Five graphs of different line types illustrate differences in angles between the sole and the ground due to differences in the walking environment of the user 4. “level ground” in the graph indicates a case where the user 4 is walking on level ground. “ascending stairs” and “descending stairs” in the graph indicate the case where the user 4 is ascending stairs and the case where the user 4 is descending stairs, respectively. “ascending a slope” and “descending a slope” in the graph indicate the case where the user 4 is ascending a slope and the case where the user 4 is descending a slope, respectively.

As can be understood from FIG. 7, the differences between the five graphs are significantly large particularly in the vicinity of the time of leaving and the vicinity of the time of landing. Therefore, the walking environment of the user 4 can be determined by extracting the feature amount from the vicinity at the time of leaving and the vicinity at the time of landing in the time series data of the angle. Specific examples of the feature amount extracted in this process include an angle at the time of leaving, an angle at the time of landing, a difference between an angle at the time of leaving and an angle at the time of landing, and a time (leaving time) from the time of leaving to the time of landing. The feature amount extracted in this process may include a plurality of elements, in other words, the feature amount extracted in this process may be a feature amount vector.

The range of time used for extracting the feature amount is preferably a period including time t1−0.02T to t1+0.02T, where T is the length of one walking cycle of the user 4 and t1 is the time at the time of landing. This is because the differences between the five graphs are particularly significant in this range. In FIG. 7, since the time t1 at the time of landing corresponds to the normalized time 70, the above range is between the normalized time 68 and 72.

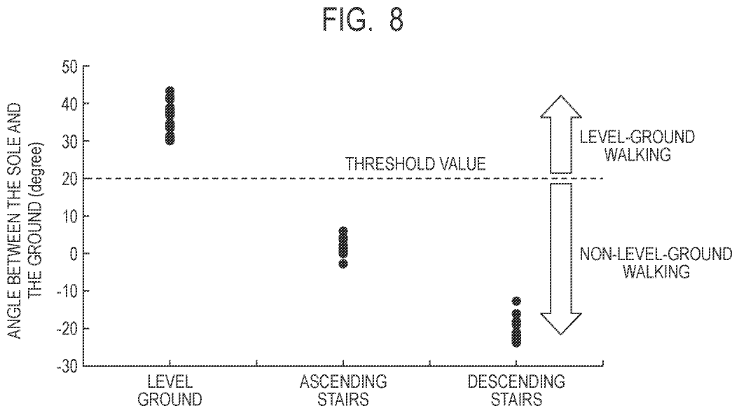

FIG. 8 is a graph illustrating an example of the angle between the sole and the ground at the time of landing. The angle values illustrate in FIG. 8 are average values between normalized times 68 to 72. As illustrated in FIG. 8, the angle between the sole and the ground is significantly different between the case where the user walks on level ground and the case where the user walks on non-level-ground place such as stairs. As described above, the angle at the time of landing is effective as a feature amount for determining whether level-ground walking or non-level-ground walking. For example, the walking environment can be determined based on the angle at the time of landing by a method in which the walking environment is determined to be the level-ground walking in a case where the angle between the sole and the ground is larger than a threshold value of 20° and the walking environment is determined to be the non-level-ground walking in a case where the angle between the sole and the ground is equal to or less than the threshold value of 20°.

In step S106, the walking environment determination unit 140 determines the walking environment based on the extracted feature amount. Thus, the walking environment determination unit 140 can determine a walking environment such as whether a location where the user 4 is walking on level ground or a location other than level ground such as stairs or a slope.

In the process of determining the walking environment from the feature amount performed by the walking environment determination unit 140, a trained model generated in advance by machine learning and stored in the storage unit 150 is used. Examples of algorithms used for machine learning include decision trees, random forests, support vector machines, neural networks, deep learning, logistic regression, k-nearest neighbor algorithm (K-NN), ensemble learning for classification method, discriminant analysis, or the like. Further, generation of a trained model by machine learning (training process) is performed in the walking environment determination device 1, the information communication terminal 2, or the server 3 using sample data prepared in advance.

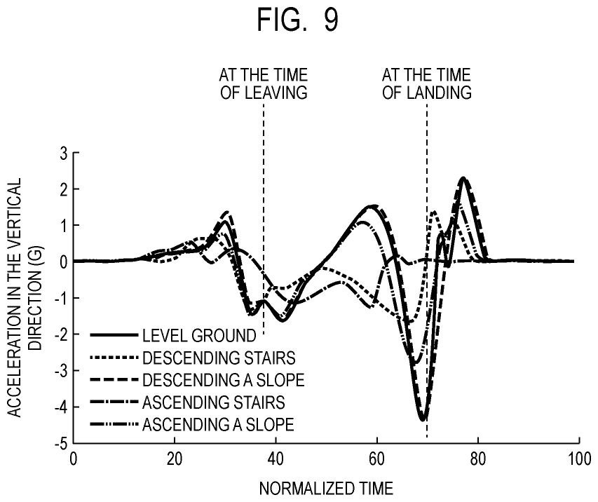

In step S105, a feature amount may be further extracted from time series data other than the angle between the sole and the ground. For example, the feature amount may be extracted from time series data of angular velocity in three axial directions or acceleration in three directions before conversion by the Madgwick filter. FIG. 9 is a graph illustrating an example of time series data of acceleration in the vertical direction within one walking cycle. The horizontal axis of FIG. 9 represents the normalized time in one walking cycle, and the vertical axis of FIG. 9 represents the acceleration in the vertical direction. The unit G illustrated in FIG. 9 is a unit of acceleration based on the standard gravitational acceleration (about 9.8 m/s²).

Since the meanings of the five graphs are the same as those in FIG. 7, description thereof is omitted. As can be understood from FIG. 9, the differences between the five graphs are significant particularly in the vicinity of the time of leaving and the vicinity of the time of landing. Therefore, the accuracy of the determination of the walking environment of the user 4 can be improved by further extracting and using the feature amount from the vicinity at the time of leaving, the vicinity at the time of landing, and the like in the time series data of the acceleration.

The training process for generating a trained model used for a determination of a walking environment in step S106 is described in more detail. This process is performed in advance in the walking environment determination device 1, the information communication terminal 2, or the server 3 prior to the process of FIG. 5. In the description of the present example embodiment, it is assumed that the training process is performed in the server 3.

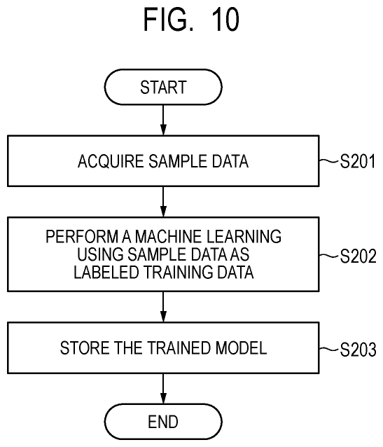

FIG. 10 is a flowchart illustrating an example of training process performed by the server 3 according to the present example embodiment. The process of FIG. 10 is performed prior to the walking state determination process at the time of shipment from a factory, calibration before the user 4 uses the walking environment determination device 1, or the like.

In step S201, the server 3 acquires sample data for training. This sample data may be, for example, one in which a label indicating a walking state is associated with a feature amount vector acquired by the processing from step S101 to step S105. The label indicating the walking state is attached in advance by the user 4, the administrator of the walking environment determination system, or the like. More specifically, by causing the user 4 to actually walk on various places such as level ground and a slope and the walking environment determination device 1 to acquire data and input the walking place, sample data in which the feature amount vector and the label are associated with each other can be created.

In step S202, the server 3 performs machine learning on the sample data as labeled training data. As a result, a trained model is generated in which an appropriate walking state is output with respect to the input of the feature amount vector.

In step S203, the server 3 stores the trained model in the flash memory 204. Thereafter, the server 3 provides the trained model to the walking environment determination device 1. Specifically, the server 3 transmits the trained model to the information communication terminal 2. The information communication terminal 2 causes the walking environment determination device 1 to install the received trained model as software for processing in the walking environment determination unit 140.

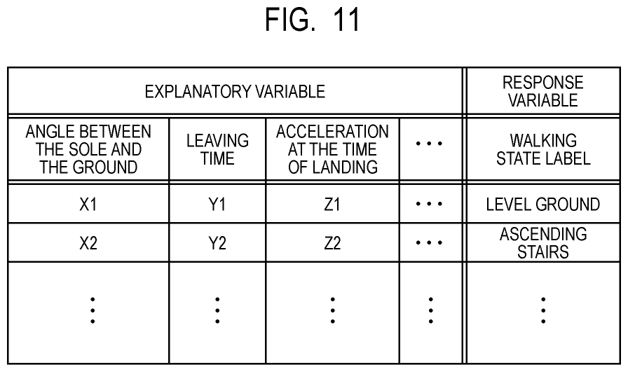

FIG. 11 is a table schematically illustrating the correspondence relation between a feature vector and a walking state label acquired by the present training process. As illustrated in FIG. 11, a walking state label such as “level ground” and “ascending stairs” is determined corresponding to a feature amount vector including “angle between the sole and the ground”, “leaving time”, “acceleration at the time of landing”, and the like. In other words, the trained model acquired by the training process has a function of outputting a walking state label as a response variable when a feature amount vector is input as an explanatory variable. It is desirable that the training process generate a trained model for each subject of walking environment determination. This is because the contents of the trained model are usually different for each subject due to a habit during walking or the like.

### Example

A result of actually determining the walking environment using the walking environment determination system of the first example embodiment is described as an example. In the present example, motion information during walking was measured for four subjects, and walking data including five types of walking environments of “level ground”, “ascending stairs”, “descending stairs”, “ascending a slope”, and “descending a slope” were acquired. A large number of feature amount vectors were extracted from these walking data to create data groups for training and validation. In the present example, cross-validation was performed using this data group. Specifically, randomly selected some data in the data group was used as validation data, and the remaining data was used as training data. That is, a trained model was generated using training data of some data group, and the recognition rate of the trained model was validated using the remaining data. The machine learning algorithm used in the present example was random forest.

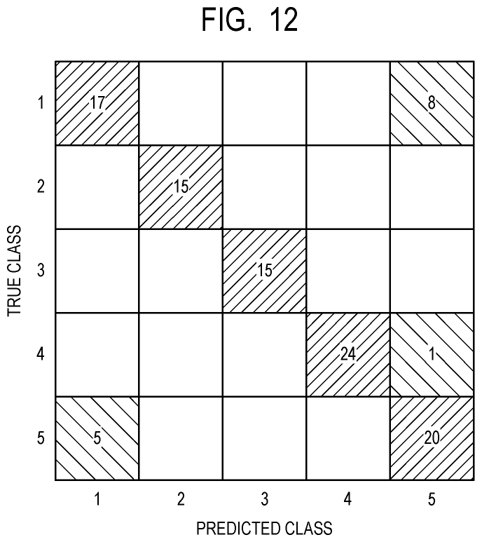

FIG. 12 is a table illustrating a result of cross-validation using this data group. The “predicted class” in the table is a class of the walking environment determined by the walking environment determination system of the first example embodiment, and the “true class” is a class indicating the walking environment of a place where the subject actually walks. The numbers “1”, “2”, “3”, “4”, and “5” of the classes in the table indicate “level ground”, “ascending stairs”, “descending stairs”, “ascending a slope”, and “descending a slope”, respectively. For example, in the prediction performed by the walking environment determination system for 25 data groups whose true class is “4 (ascending a slope)”, the walking environment determination system correctly predicted the class “4” for 24 out of 25 data groups. In contrast, an incorrect class “5” was predicted for one of 25 data groups. As illustrated in FIG. 12, in the walking environment system of the first example embodiment, the walking environment could be determined at a high correct rate of about 87%. As described above, according to the present example embodiment, the walking environment determination device 1 and the walking environment determination system capable of determining the walking environment with high accuracy are provided.

As described above, according to the present example embodiment, the information processing device 11 capable of suitably extracting the feature amount used for determining the walking environment is provided. Further, the walking environment determination device 1 and the walking environment determination system capable of determining the walking environment with high accuracy are provided by using the feature amount extracted by the information processing device 11.

The walking environment determination system of the present example embodiment can determine whether or not the location where the user 4 is walking is level ground. Hereinafter, an example of application of the walking environment determination system of the present example embodiment is described.

Generally, the walking pattern of a human is different between level ground and ground other than the level ground. Therefore, when the analysis of the walking pattern of the user 4 (gait analysis) is performed, the walking data of a place other than the level ground such as a slope or stairs may be excluded. In such a case, there is a need to determine from the walking data whether or not the location where the user 4 is walking is the level ground. By determining the walking environment using the walking environment determination system of the present example embodiment, the walking data of a place other than the level ground can be easily excluded from the walking data for gait analysis. In addition, the storage capacity and the communication amount of the walking data can be reduced by performing the data processing for deleting the walking data of a place other than the level ground at the time of acquiring the walking data. Alternatively, the power consumption of the walking environment determination device 1 can be reduced by controlling the IMU 12 so as not to acquire walking data of a place other than the level ground.

### Second Example Embodiment

A walking environment determination system of the present example embodiment is different from that of the first example embodiment in that the walking environment determination system has a function of measuring walking data for gait analysis and a function of switching two modes with different power consumption. Hereinafter, differences from the first example embodiment are mainly described, and description of common portions is omitted or simplified.

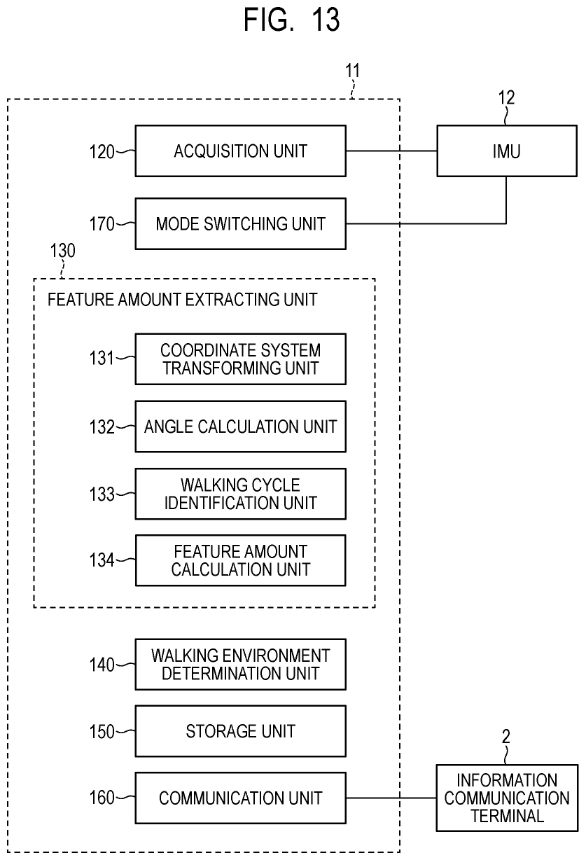

FIG. 13 is a functional block diagram of the information processing device 11 according to the present example embodiment. The information processing device 11 further includes a mode switching unit 170 in addition to the elements described in the first example embodiment. The IMU 12 of the present example embodiment can operate in a normal mode and a power saving mode in which power consumption thereof is smaller than that in the normal mode. The mode switching unit 170 has a function of controlling the operation mode of the IMU 12 to the normal mode or the power saving mode. The normal mode may be referred to as the first mode more generally, and the power saving mode may be referred to as the second mode more generally.

The difference between the normal mode and the power saving mode may be a difference in types of processes that the IMU 12 can perform. For example, the power saving mode may reduce power consumption by stopping functions of some devices in the IMU 12. Alternatively, the difference between the normal mode and the power saving mode may be a difference in sampling rate. In this case, the power consumption of the sensors in the IMU 12 can be reduced by reducing the frequency of acquiring data in the power saving mode as compared with the normal mode.

The CPU 111 realizes the function of the mode switching unit 170 by loading a program stored in the ROM 113, the flash memory 114, or the like into the RAM 112 and executing the program. The function of the mode switching unit 170 may be provided in the information communication terminal 2 or the server 3, or may be provided in the IMU 12.

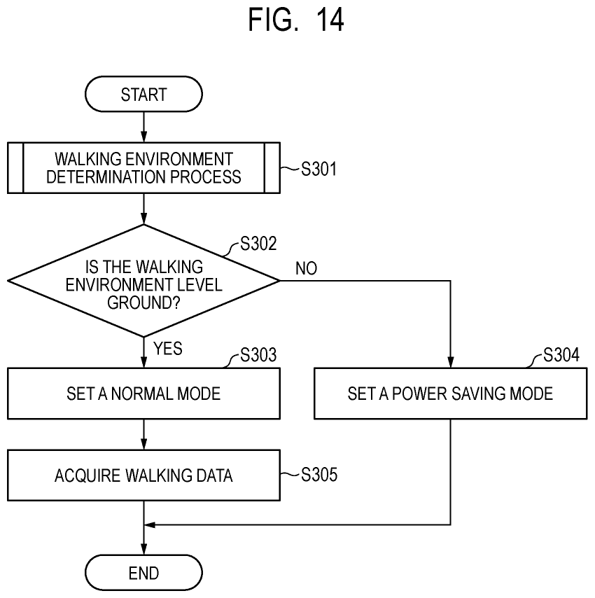

FIG. 14 is a flowchart illustrating an example of a mode switching process performed by the walking environment determination device 1 according to the present example embodiment. The process of FIG. 14 is performed when the walking environment determination device 1 detects walking, for example, when the user 4 is walking. Alternatively, the process of FIG. 14 may be always performed unrelated to whether or not the user 4 is walking, or may be performed at predetermined time intervals.

In step S301, each unit of the walking environment determination device 1 performs walking environment determination process. This walking environment determination process is the same as steps S101 to S106 in FIG. 5, and a description thereof is omitted.

In step S302, the mode switching unit 170 determines whether or not the walking environment of the user 4 is level ground based on the result of the walking environment determination process. If it is determined that the walking environment is level ground (YES in step S302), the process proceeds to step S303. If it is determined that the walking environment is not level ground (NO in step S302), the process proceeds to step S304.

In step S303, the mode switching unit 170 controls the operation mode of the IMU 12 to the normal mode. Thereafter, in step S305, the acquisition unit 120 controls the IMU 12 to acquire walking data for gait analysis.

In step S304, the mode switching unit 170 controls the operation mode of the IMU 12 to the power saving mode, and the process ends. When the walking data for gait analysis can be acquired even in the power saving mode, the same processing as in step S305 may be performed after step S304.

In order to properly perform gait analysis, it is desirable to use walking data in which the walking environment is level ground. In contrast, walking data acquired in a walking environment other than level ground is often not so effective for gait analysis. Therefore, the information processing device 11 of the present example embodiment can acquire walking data for gait analysis in the normal mode when the walking environment is level ground, and reduces power consumption by switching to the power saving mode when the walking environment is not level ground. Therefore, it is possible to achieve both appropriate acquisition of walking data and low power consumption.

As described above, the information processing device 11 of the present example embodiment can reduce power consumption by controlling the IMU 12 to be in the power saving mode when the walking environment is not level ground, in addition to the same effects as those of the first example embodiment. In a case where the IMU 12 is provided in the shoe 5 or the like of the user 4 as in the present example embodiment, the power capacity of the battery 13 for driving the IMU 12 cannot be increased so much. Therefore, reduction in power consumption according to the present example embodiment is effective.

The device or system described in the above example embodiments can also be configured as in the following third example embodiment.

### Third Example Embodiment

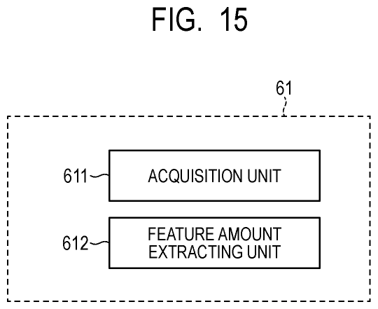

FIG. 15 is a functional block diagram of the information processing device 61 according to the third example embodiment. The information processing device 61 includes an acquisition unit 611 and a feature amount extracting unit 612. The acquisition unit 611 acquires motion information of a foot of a user measured by a motion measurement device provided on the foot. The feature amount extracting unit 612 extracts a feature amount indicating a walking environment from an angle between a sole and a ground generated based on the motion information.

According to the present example embodiment, there is provided an information processing device 61 capable of suitably extracting a feature amount used for determining a walking environment. [Modified Example embodiments]

The present invention is not limited to the example embodiments described above, and may be suitably modified within the scope of the present invention. For example, an example in which a part of the configuration of one example embodiment is added to another example embodiment or an example in which a part of the configuration of one example embodiment is replaced with another example embodiment is also an example embodiment of the present invention.

In the above-described example embodiment, the motion measurement device including the angular velocity sensor that measures the angular velocity in the three axial directions and the acceleration sensor that measures the acceleration in the three directions is used, but sensors other than these may also be used. For example, a magnetic sensor that detects geomagnetism by detecting magnetism in three directions to identify an azimuth may be further used. Even in this case, the same processing as the above-described example embodiment can be applied, and the accuracy can be further improved.

Although the walking environment determination process is performed inside the walking environment determination device 1 in the above-described example embodiments, this function may be provided in the information communication terminal 2. In this case, the information communication terminal 2 functions as a walking environment determination device.

A processing method in which a program for operating the configuration of the above-described example embodiments is recorded in a storage medium so as to implement the functions of the above-described example embodiments, the program recorded in the storage medium is read as code, and the program is executed in a computer is also included in the scope of each example embodiment. That is, a computer-readable storage medium is also included in the scope of the example embodiments. Further, not only the storage medium in which the above program is recorded, but also the program itself is included in each example embodiment. In addition, one or more components included in the above-described example embodiments may be a circuit such as an application specific integrated circuit (ASIC), a field programmable gate array (FPGA) configured to implement the functions of each component.

As the storage medium, for example, a floppy (registered trademark) disk, a hard disk, an optical disk, a magneto-optical disk, a compact disk (CD)-ROM, a magnetic tape, a nonvolatile memory card, or a ROM can be used. Further, the scope of each example embodiment is not limited to the case where the processing is executed by the program alone recorded in the storage medium, and a case where the processing is executed by operating on an operating system (OS) in cooperation with the functions of other software and extension board is also included in the scope of each example embodiment.

The service realized by the functions of the above-described example embodiments may be provided to the user in the form of a software as a service (SaaS).

It should be noted that the above-described example embodiments are merely examples of embodying the present invention, and the technical scope of the present invention should not be limitedly interpreted by these. That is, the present invention can be implemented in various forms without departing from the technical idea or the main features thereof.

The whole or part of the example embodiments disclosed above can be described as, but not limited to, the following supplementary notes.

### (Supplementary Note 1)

An information processing device comprising: an acquisition unit configured to acquire motion information of a foot of a user measured by a motion measurement device provided on the foot; and a feature amount extracting unit configured to extract a feature amount indicating a walking environment from an angle between a sole and a ground generated based on the motion information.

### (Supplementary Note 2)

The information processing device according to supplementary note 1, wherein the motion information includes time series data of at least one walking cycle.

### (Supplementary Note 3)

The information processing device according to supplementary note 1 or 2, wherein the motion information includes acceleration and angular velocity.

### (Supplementary Note 4)

The information processing device according to supplementary note 3, wherein the feature amount extracting unit includes a coordinate transforming unit configured to transform a coordinate system of the acceleration and the angular velocity included in the motion information into a coordinate system with respect to the foot.

### (Supplementary Note 5)

The information processing device according to supplementary note 3 or 4, wherein the feature amount extracting unit includes an angle calculation unit configured to calculate the angle using the acceleration and the angular velocity.

### (Supplementary Note 6)

The information processing device according to supplementary note 5, wherein the angle calculation unit calculates the angle using a Madgwick filter.

### (Supplementary Note 7)

The information processing device according to any one of supplementary notes 3 to 6, wherein the feature amount extracting unit further extracts a feature amount indicating a walking environment from the acceleration or the angular velocity.

### (Supplementary Note 8)

The information processing device according to any one of supplementary notes 1 to 7, wherein the motion measurement device is provided at a position corresponding to an arch of the foot.

### (Supplementary Note 9)

The information processing device according to any one of supplementary notes 1 to 8, wherein the feature amount extracting unit extracts the feature amount based on the angle of a period including at least a moment at which the foot lands.

### (Supplementary Note 10)

The information processing device according to any one of supplementary notes 1 to 9,

wherein the feature amount extracting unit extracts the feature amount based on the angle of a period from t1−0.02T to t1+0.02T,

where T represents a length of the one walking cycle of the user and t1 represents a time of the moment at which the foot lands.

### (Supplementary Note 11)

The information processing device according to any one of supplementary notes 1 to 10, wherein the feature amount is used to determine whether or not the walking environment of the user is level ground.

### (Supplementary Note 12)

The information processing device according to supplementary note 11,

wherein the motion measurement device is operable in a first mode and a second mode, power consumption thereof in the second mode being smaller than that in the first mode,

wherein the motion measurement device operates in the first mode in a case where it is determined that the walking environment of the user is level ground, and

wherein the motion measurement device operates in the second mode in a case where it is determined that the walking environment of the user is not level ground.

### (Supplementary Note 13)

A walking environment determination device configured to determine a walking environment of a user based on a feature amount extracted by the information processing device according to any one of supplementary notes 1 to 12.

### (Supplementary Note 14)

A walking environment determination system comprising:

the information processing device according to any one of supplementary notes 1 to 12;

a walking environment determination device configured to determine the walking environment of the user based on the feature amount; and the motion measurement device.

### (Supplementary Note 15)

An information processing method comprising:

acquiring motion information of a foot of a user measured by a motion measurement device provided on the foot; and

extracting a feature amount indicating a walking environment from an angle between a sole and a ground generated based on the motion information.

### (Supplementary Note 16)

A storage medium storing a program that causes a computer to perform:

acquiring motion information of a foot of a user measured by a motion measurement device provided on the foot; and

extracting a feature amount indicating a walking environment from an angle between a sole and a ground generated based on the motion information.

### REFERENCE SIGNS LIST

1 walking environment determination device
2 information communication terminal
3 server
4 user
5 shoe
11, 61 information processing device
12 IMU
13 battery
111, 201 CPU
112, 202 RAM
113, 203 ROM
114, 204 flash memory
115, 205 communication I/F
116 IMU control device
120, 611 acquisition unit
130, 612 feature amount extracting unit
131 coordinate system transforming unit
132 angle calculation unit
133 walking cycle identification unit
134 feature amount calculation unit
140 walking environment determination unit
150 storage unit
160 communication unit
170 mode switching unit
206 input device
207 output device

What is claimed is:

1. An information processing device comprising:
a memory configured to store instructions; and
a processor configured to execute the instructions to:
acquire motion information of a foot of a user measured by a motion measurement device provided on the foot; and
extract a feature amount indicating a walking environment from an angle between a sole and a ground generated based on the motion information.

2. The information processing device according to claim 1, wherein the motion information includes time series data of at least one walking cycle.

3. The information processing device according to claim 1, wherein the motion information includes acceleration and angular velocity.

4. The information processing device according to claim 3, wherein the processor is further configured to execute the instructions to transform a coordinate system of the acceleration and the angular velocity included in the motion information into a coordinate system with respect to the foot.

5. The information processing device according to claim 3, the processor is further configured to execute the instructions to calculate the angle using the acceleration and the angular velocity.

6. The information processing device according to claim 5, wherein the angle is calculated using a Madgwick filter.

7. The information processing device according to claim 3, wherein a feature amount indicating a walking environment is further extracted from the acceleration or the angular velocity.

8. The information processing device according to claim 1, wherein the motion measurement device is provided at a position corresponding to an arch of the foot.

9. The information processing device according to claim 1, wherein the feature amount is extracted based on the angle of a period including at least a moment at which the foot lands.

10. The information processing device according to claim 1,
wherein the feature amount is extracted based on the angle of a period from t1−0.02T to t1+0.02T,
where T represents a length of the one walking cycle of the user and t1 represents a time of the moment at which the foot lands.

11. The information processing device according to claim 1, wherein the feature amount is used to determine whether or not the walking environment of the user is level ground.

12. The information processing device according to claim 11,
wherein the motion measurement device is operable in a first mode and a second mode, power consumption thereof in the second mode being smaller than that in the first mode,
wherein the motion measurement device operates in the first mode in a case where it is determined that the walking environment of the user is level ground, and
wherein the motion measurement device operates in the second mode in a case where it is determined that the walking environment of the user is not level ground.

13. A walking environment determination device configured to determine a walking environment of a user based on a feature amount extracted by the information processing device according to claim 1.

14. A walking environment determination system comprising:
the information processing device according to claim 1;
a walking environment determination device configured to determine the walking environment of the user based on the feature amount; and
the motion measurement device.

15. An information processing method comprising:
acquiring motion information of a foot of a user measured by a motion measurement device provided on the foot; and
extracting a feature amount indicating a walking environment from an angle between a sole and a ground generated based on the motion information.

16. A non-transitory storage medium storing a program that causes a computer to perform:
acquiring motion information of a foot of a user measured by a motion measurement device provided on the foot; and
extracting a feature amount indicating a walking environment from an angle between a sole and a ground generated based on the motion information.
